(19) United States
(12) Patent Application Publication
(10) Pub. No.: US 2024/0309128 A1
(43) Pub. Date: Sep. 19, 2024

(54) PERFLUOROPYRIDINE BASED POLYMERS OBTAINED VIA RADICAL INITIATED METHODS

(71) Applicant: Government of the United States, as represented by the Secretary of the Air Force, Wright-Patterson AFB, OH (US)

(72) Inventors: Abby R. Jennings, Colorado Springs, CO (US); Scott T. Iacono, Colorado Springs, CO (US); Zachary J. Auleciems, N Lake Elmo, MN (US); Emeline C. Lochmaier, Colorado Springs, CO (US)

(21) Appl. No.: 18/598,136

(22) Filed: Mar. 7, 2024

Related U.S. Application Data
(60) Provisional application No. 63/550,141, filed on Feb. 6, 2024.
(60) Provisional application No. 63/451,658, filed on Mar. 13, 2023.

(51) Int. Cl. C08F 26/06; C07D 213/26; C07D 213/61

(52) U.S. Cl. CPC C08F 26/06; C07D 213/74; C08F 2438/02

(57) ABSTRACT

The synthesis of the fluorinated monomer, 2-((perfluoropyridin-4-yl)amino)ethyl methacrylate, and the use of the same in the preparation of fluorinated homo- and copolymers is provided. Such monomer was designed to contain a terminal methacrylate group, allowing for polymerization through free-radical techniques, both chain-growth and reversible addition-fragmentation chain-transfer methods. The monomer was designed to be compatible with other commercially available monomers and solvents to facilitate ease of copolymerization through free-radical polymerization techniques. The polymerization does not require perfluorinated alkyl substances or perfluorooctanoic acids or related compounds in the synthesis of the fluoropolymers.

### CROSS-REFERENCE TO RELATED APPLICATION

The present application claims priority to U.S. Provisional Application Ser. No. 63/550,141 filed Feb. 6, 2024, and U.S. Provisional Application Ser. No. 63/451,658 filed Mar. 13, 2023, the contents of both such provisional applications hereby being incorporated by reference in their entry.

### RIGHTS OF THE GOVERNMENT

The invention described herein may be manufactured and used by or for the Government of the United States for all governmental purposes without the payment of any royalty.

### FIELD OF THE INVENTION

The present invention relates to perfluoropyridine based polymers obtained via radical initiated methods and methods of making and using articles comprising such polymers.

### BACKGROUND OF THE INVENTION

Fluorinated polymers, or fluoropolymers (FPs) have found use in a wide range of advanced applications, including but not limited to, the medical field, the automotive and aerospace industries, and advanced coatings and surfaces. The motivation behind this, is the unique properties inherent in these highly fluorinated materials. These properties consist of low flammability, water absorptivity, surface energy, and refractive indexes. Furthermore, FPs demonstrate increased mechanical strength, chemical resistance, and thermal stability. These highly sought-after properties can be assigned to the high fluorine content, in the form of C—F bonds, of the FPs.

Previous efforts to obtain FPs, have relied on the incorporation of perfluorinated alkyl chains or perfluorinated alkyl substances (PFAS), either in the backbone of the polymer or as a side chain. PFAS are classified as being aliphatic compounds of the general formula of CₙF₂ₙ₊₁. Furthermore, perfluorooctanoic acid (PFOA) and related compounds have been, and some are still being used, as polymerization aids for the preparation of FPs. PFOAs have also been found as by-products and/or degradation products in the synthesis of FPs. Both PFAS and PFOAs have been classified as forever chemicals, are known to bioaccumulate, cause adverse health effects, and are harmful to the environment. With regulations on the use of PFOAs and PFAS gaining traction in the United States, European Union, and beyond, there is a need to develop alternative technologies for obtaining FPs. One potential opportunity for accomplishing this is by the use of perfluorinated aromatics, such as perfluroropyridine (PFPy), to prepare monomers and FPs. Although the use of PFPy in the preparation of FPs is not novel, there are no known examples where PFPy is used to prepare a monomer that can be used to prepare linear, processable FPs via free-radical initiated pathways.

### SUMMARY OF THE INVENTION

The synthesis of the fluorinated monomer, 2-((perfluoropyridin-4-yl)amino)ethyl methacrylate, and the use of the same in the preparation of fluorinated homo- and copolymers is provided. Such monomer was designed to contain a terminal methacry late group, allowing for polymerization through free-radical techniques, both chain-growth and reversible addition-fragmentation chain-transfer methods. The monomer was designed to be compatible with other commercially available monomers and solvents to facilitate ease of copolymerization through free-radical polymerization techniques. The polymerization does not require perfluorinated alkyl substances or perfluorooctanoic acids or related compounds in the synthesis of the fluoropolymers.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1A shows the ¹H NMR spectra for Poly 1-4 collected at 60 mg/ml of each polymer in THF-d₈.

FIG. 1B shows the ¹⁹F NMR spectra for Poly 1-4 collected at 60 mg/ml of each polymer in THF-d₈.

FIG. 2 shows the FTIR Spectrum of Poly 1-4.

FIG. 3A shows the ¹H NMR spectra for Poly 5-8 collected at 15 mg/mL of each polymer in acetone-d₆.

FIG. 3B shows the ¹⁹F NMR spectra for Poly 5-8 collected at 15 mg/ml of each polymer in acetone-d₆.

FIG. 4 shows the FTIR spectrum of Poly 5-8.

FIG. 5A shows the ¹H NMR spectra for Poly 9-12 collected at 15 mg/ml of each polymer in acetonitrile-d₃.

FIG. 5B shows the ¹⁹F NMR spectra for Poly 9-12 collected at 15 mg/ml of each polymer in acetonitrile-d₃.

FIG. 6 shows the FTIR spectrum of Poly 9-12.

FIG. 7A shows the. ¹H spectra for Poly 13-16 collected at 15 mg/ml of each polymer in dimethyl sulfoxide-d₆.

FIG. 7B shows the ¹⁹F NMR spectra for Poly 13-16 collected at 15 mg/mL of each polymer in dimethyl sulfoxide-d₆.

FIG. 8 shows the FTIR spectrum of Poly 13-16.

FIG. 9A shows the. ¹H spectra for Poly 17-19 collected at 15 mg/ml of each polymer in dimethyl sulfoxide-d₆.

FIG. 9B shows the ¹⁹F NMR spectra for Poly 17-19 collected at 15 mg/ml of each polymer in dimethyl sulfoxide-d₆.

FIG. 10 shows the FTIR spectrum of Poly 17-19.

FIG. 11A shows the. ¹H spectra for Poly 20-22 collected at 15 mg/ml of each polymer in dimethyl sulfoxide-d₆.

FIG. 11B shows the ¹⁹F NMR spectra for Poly 20-22 collected at 15 mg/ml of each polymer in dimethyl sulfoxide-d₆.

FIG. 12 shows the FTIR spectrum of Poly 20-22.

FIG. 13A shows the. ¹H spectra for Poly 23-26 collected at 15 mg/ml of each polymer in acetonitrile-d₆.

FIG. 13B shows the ¹⁹F NMR spectra for Poly 23-26 collected at 15 mg/ml of each polymer in acetonitrile-d₆.

FIG. 14 shows the FTIR spectrum of Poly 23-26.

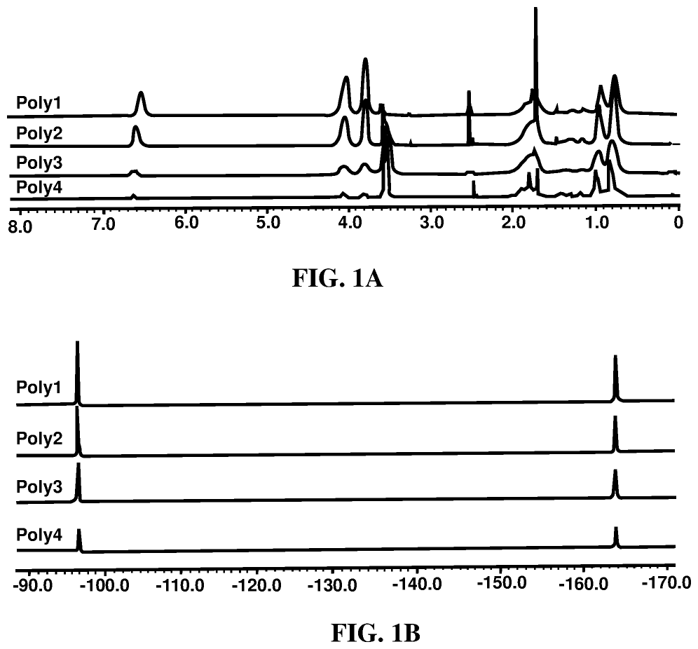

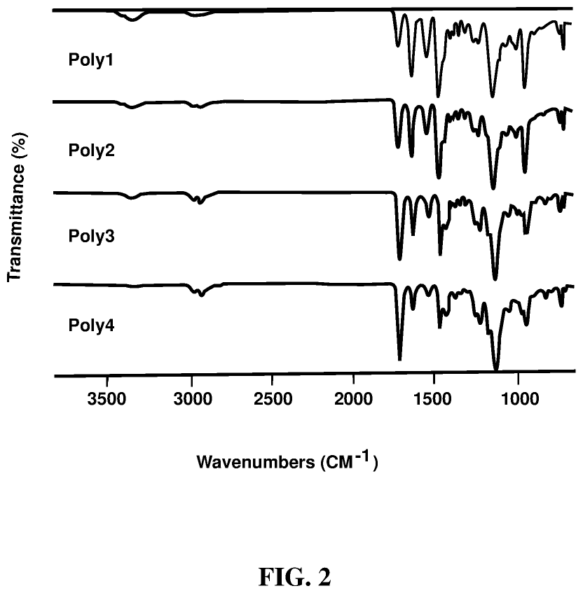

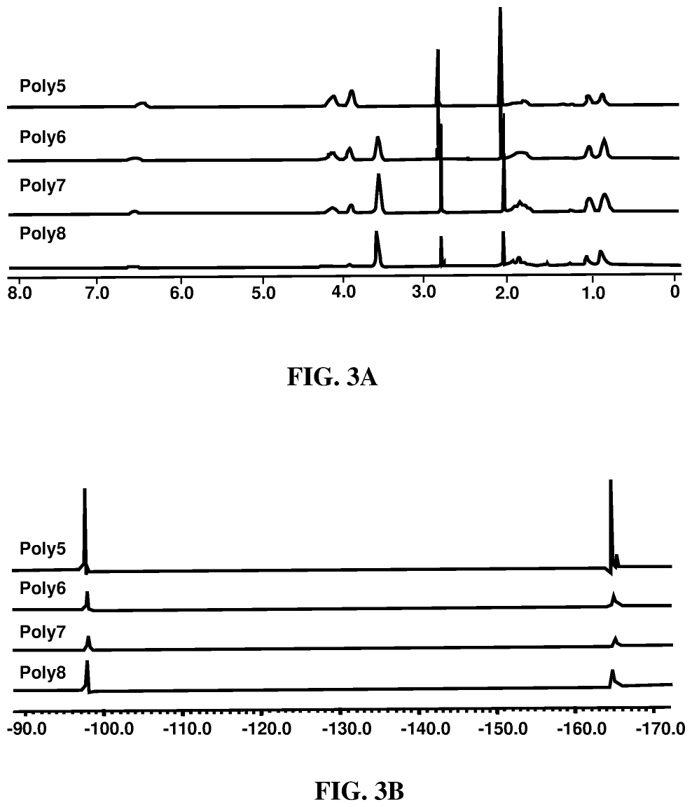

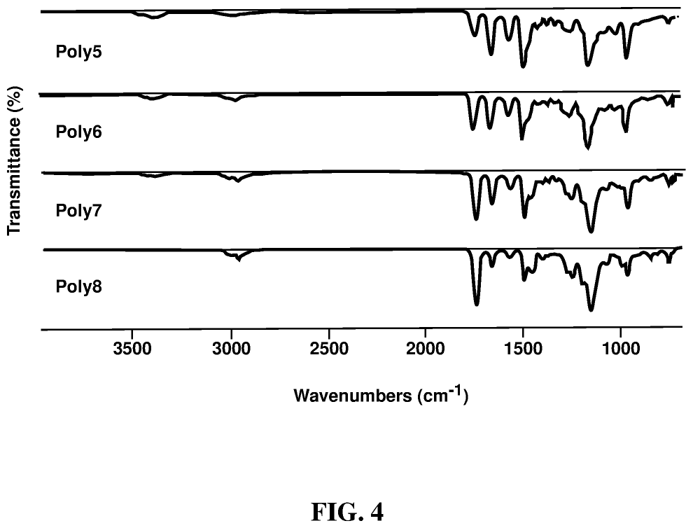

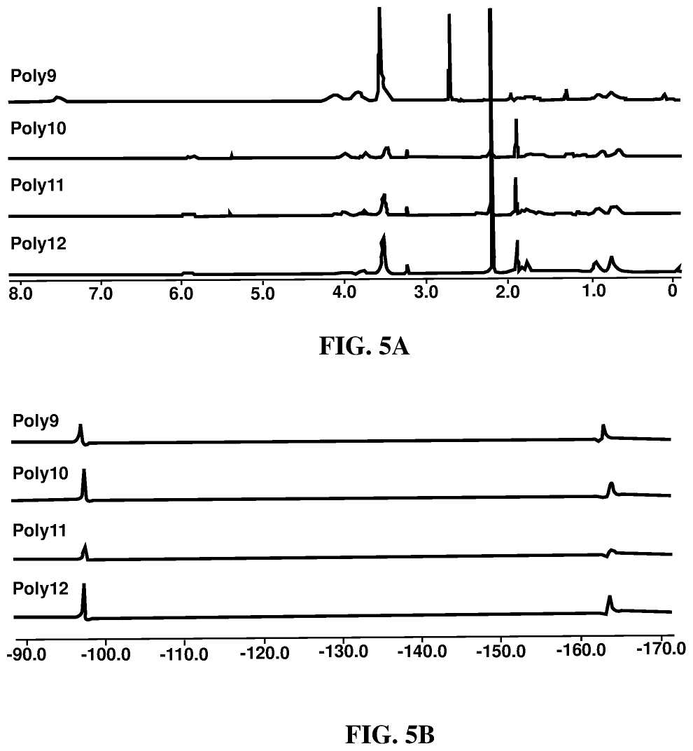

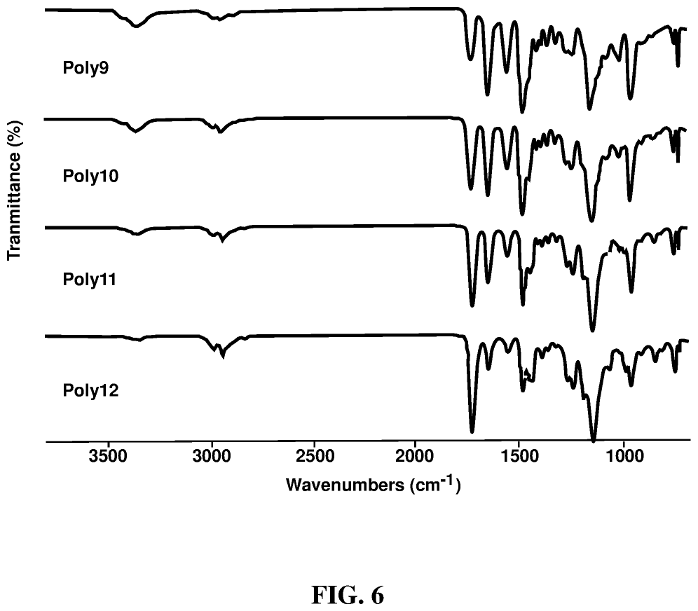

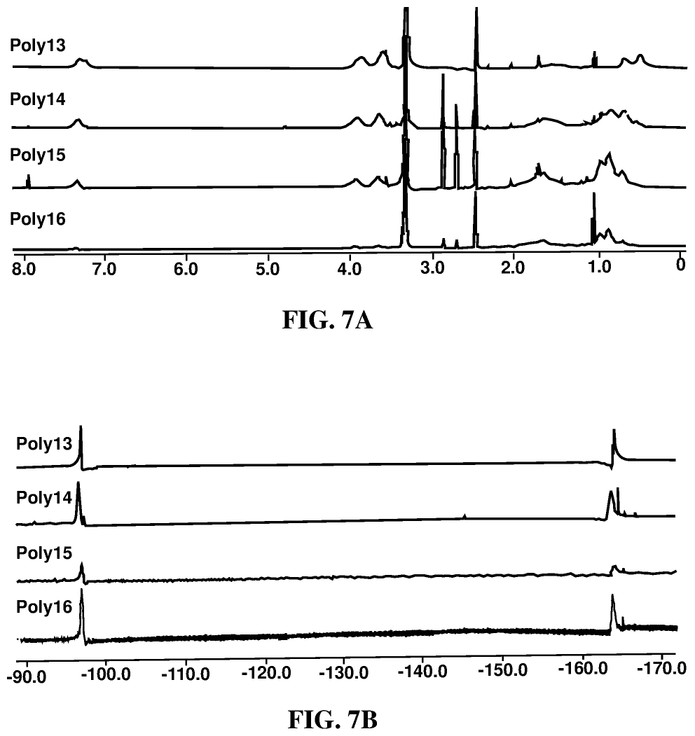

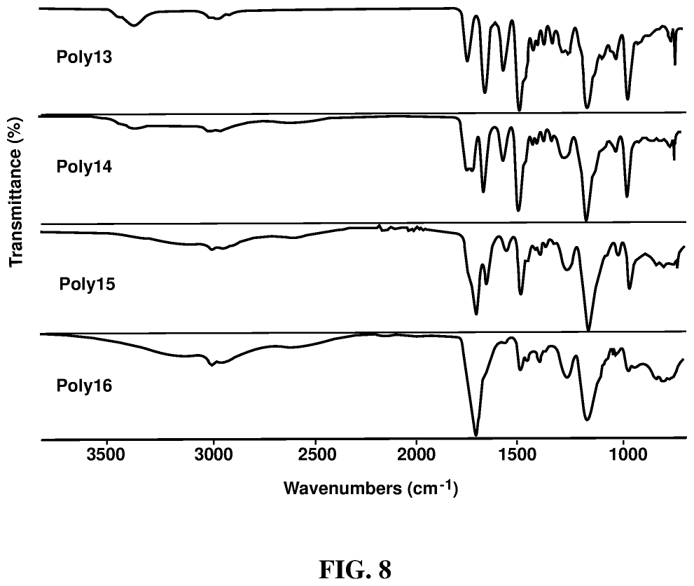

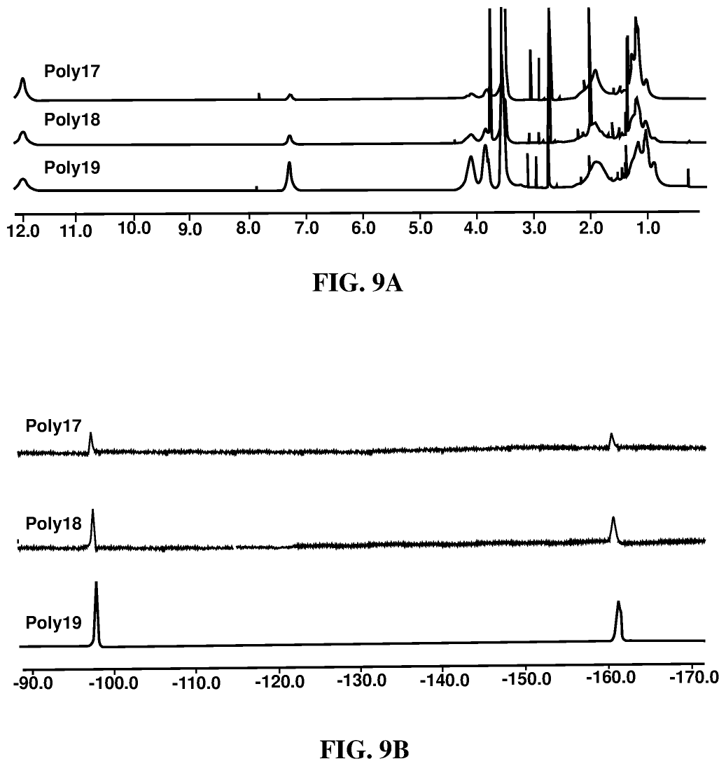

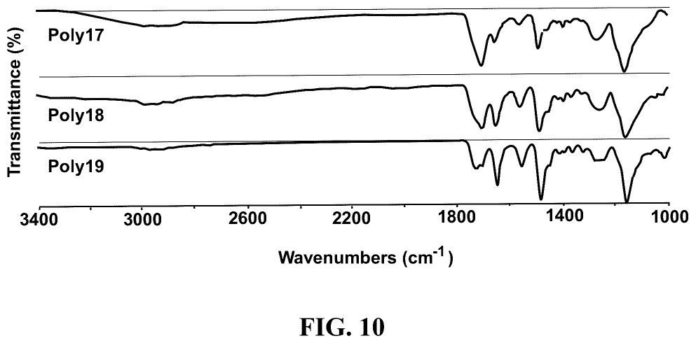

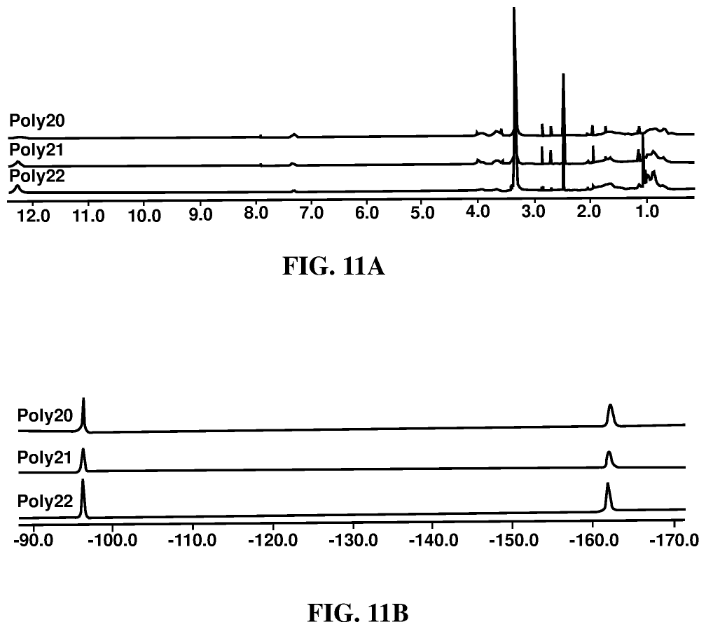

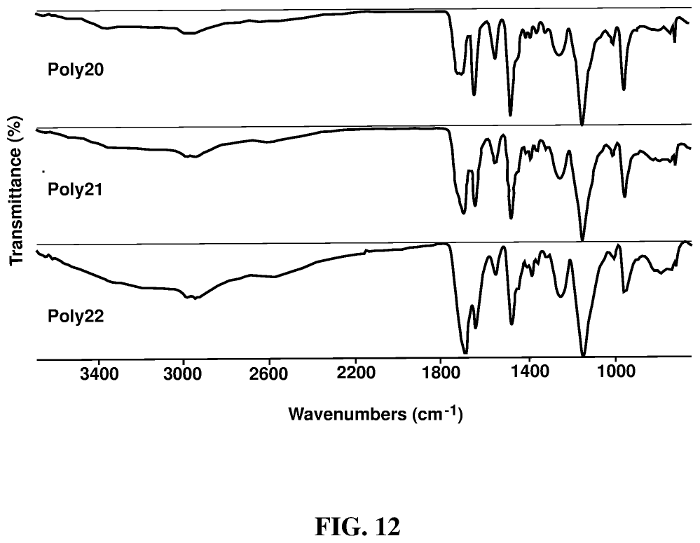

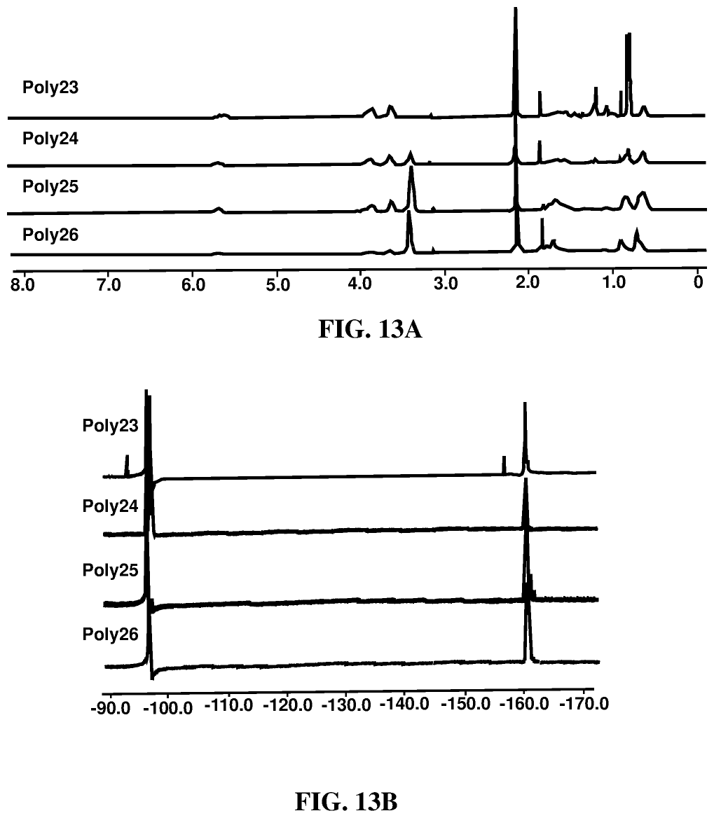

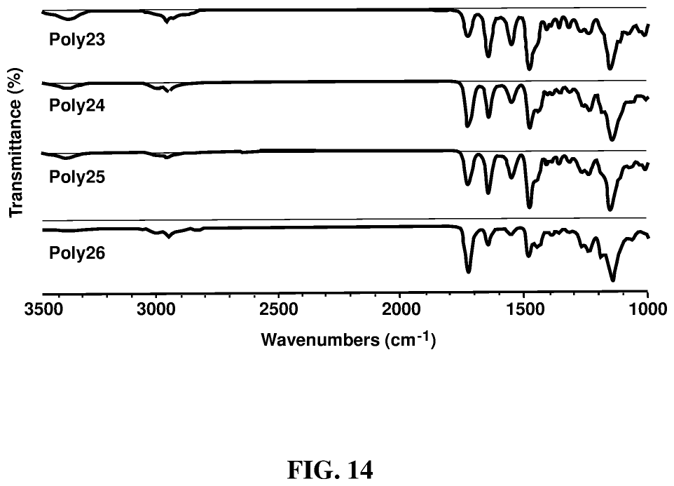

### DETAILED DESCRIPTION OF THE INVENTION

### Definitions

Unless specifically stated otherwise, as used herein, the terms “a”, “an” and “the” mean “at least one”.

As used herein, the terms “include”, “includes” and “including” are meant to be non-limiting.

As used herein, the words “about,” “approximately,” or the like, when accompanying a numerical value, are to be construed as indicating a deviation as would be appreciated by one of ordinary skill in the art to operate satisfactorily for an intended purpose.

As used herein, the words “and/or” means, when referring to embodiments (for example an embodiment having elements A and/or B) that the embodiment may have element A alone, element B alone, or elements A and B taken together.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Monomers, Polymers Comprising Said Monomers and Articles Comprising Same

Applicants disclose a monomer having the following formula:

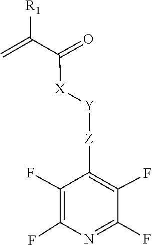

wherein R₁ is H or a hydrocarbon; X=O, NH, or S; Y=a hydrocarbon; and Z=O, NH, or S.

Applicants disclose the monomer of the previous paragraph wherein: R₁ is H, methyl, ethyl, propyl, iso-propyl, n-butyl, tert-butyl, sec-butyl, n-pentyl, n-hexyl, n-hepyl, n-octyl, or phenyl; and Y is ethyl, propyl, iso-propyl, n-butyl, tert-butyl, sec-butyl, n-pentyl, n-hexyl, n-hepyl, n-octyl, or phenyl.

Applicants disclose the monomer of the two previous paragraphs wherein R₁ is H or methyl, Y is ethyl or phenyl, X is O.

Applicants disclose a polymer comprising the monomer of the three preceding paragraphs.

Applicants disclose a polymer according to the previous paragraph, said polymer having Structure 1 or Structure 2 below:

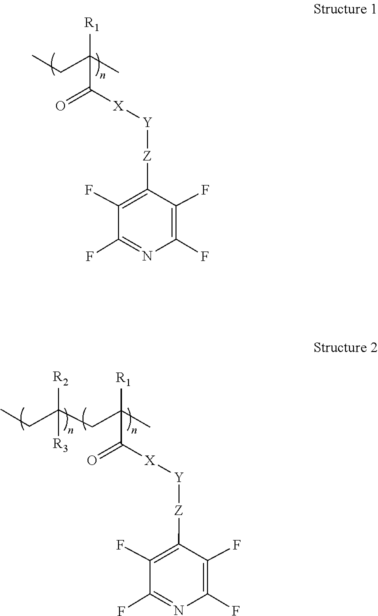

wherein for Structure 1 R₁ is H or a hydrocarbon; X is O, NH, or S; Y is a hydrocarbon; Z is O, NH, or S; and the indice n is an integer from 20 to 150; and wherein for Structure 2 R₁, R₂, or R₃ are each independently H or a hydrocarbon; X is O, NH, or S; Y is a hydrocarbon; Z is O, NH, or S; and the indice n is an integer from 20 to 150.

Applicants disclose a polymer according to the previous paragraph: wherein for Structure 1: R₁ is H, methyl, ethyl, propyl, iso-propyl, n-butyl, tert-butyl, sec-butyl, n-pentyl, n-hexyl, n-hepyl, n-octyl, or phenyl; and Y is ethyl, propyl, iso-propyl, n-butyl, tert-butyl, sec-butyl, n-pentyl, n-hexyl, n-hepyl, n-octyl, or phenyl; and wherein for Structure 2: R₁ is H, methyl, ethyl, propyl, iso-propyl, n-butyl, tert-butyl, sec-butyl, n-pentyl, n-hexyl, n-hepyl, n-octyl, or phenyl: R₂ and are R₃ are each independently H, methyl, ethyl, propyl, iso-propyl, n-butyl, tertbutyl, sec-butyl, n-pentyl, n-hexyl, n-hepyl, n-octyl, phenyl, O(C═O)CH3, O(C═O)OH, Cl, F, CN; and Y is ethyl, propyl, iso-propyl, n-butyl, tert-butyl, sec-butyl, n-pentyl, n-hexyl, n-hepyl, n-octyl, or phenyl.

Applicants disclose a polymer according to the previous paragraph wherein for Structure 1 R₁ is H or methyl, Y is ethyl or phenyl, and X is O; and for Structure 2 R₁ is H or methyl, R₂ is H or methyl, R₃ is O(C═O)CH3, Y is ethyl or phenyl, and X is O.

Applicants disclose a polymer according to the previous four paragraphs, said polymer having a number average molecular weight of from about 5,000 Da to about 50,000 Da based on a polystyrene standard and said polymer having a number average molecular weight of from about 8,000 Da to about 75,000 Da based on a polymethylmethacrylate standard.

Applicants disclose a polymer according to the previous four paragraphs, said polymer having a polydispersity of from about 1.2 to about 4.8 with respect to a polymethylmethacrylate standard.

Applicants disclose an article comprising a polymer according to the previous six paragraphs.

Applicants disclose an article according to the previous paragraph, said article being a textile, or a membrane. Benefits of including the perfluoropyridine (PFPy) unit includes additional fluorine content to improve water-and oil-repellency in addition to permeability for breathable fabrics and/or membranes.

### Process of Making Monomers and Polymers

Applicants disclose a process of making a monomer according to the previous section titled “Monomers, Polymers Comprising Said Monomers and Articles Comprising Same”, said process comprising reacting a monomeric precursor having Structure 1 and an acrylol chloride having Structure 2 in the presence of an amine base, preferably said amine base is triethylamine

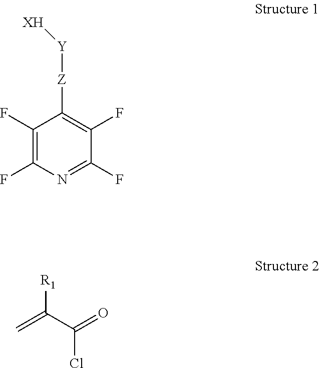

wherein for Structure 1 X is O, NH, or S; Y is a hydrocarbon; and Z is O, NH, or S; and for Structure 2 R₁ is H or a hydrocarbon.

Applicants disclose a process of making a monomer according to the previous paragraph wherein: for Structure 1 Y is ethyl, propyl, iso-propyl, n-butyl, tert-butyl, sec-butyl, n-pentyl, n-hexyl, n-hepyl, n-octyl, or phenyl: and for Structure 2 R₁ is H, methyl, ethyl, propyl, iso-propyl, n-butyl, tert-butyl, sec-butyl, n-pentyl, n-hexyl, n-hepyl, n-octyl, or phenyl.

Applicants disclose a process of making a monomer according to the according to the previous paragraph wherein for Structure 1 Y is ethyl or phenyl, X is O; and for Structure 2 R₁ is H or methyl.

Applicants disclose a process of making a polymer according to the previous section titled “Monomers, Polymers Comprising Said Monomers and Articles Comprising Same”, said process comprising polymerizing a monomer having Structure 3 below:

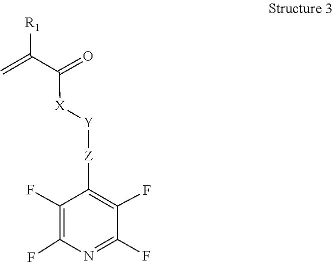

wherein R₁ is H or a hydrocarbon; X is O, NH, or S; Y is a hydrocarbon; and Z is O, NH, or S.

Applicants disclose a process of making a polymer according to the previous paragraph wherein for said monomer having Structure 3: R₁ is H, methyl, ethyl, propyl, iso-propyl, n-butyl, tert-butyl, sec-butyl, n-pentyl, n-hexyl, n-hepyl, n-octyl, or phenyl; and Y is ethyl, propyl, iso-propyl, n-butyl, tert-butyl, sec-butyl, n-pentyl, n-hexyl, n-hepyl, n-octyl, or phenyl.

Applicants disclose a process of making a polymer according to the previous paragraph wherein for said monomer having Structure 3 R₁ is H or methyl, Y is ethyl or phenyl, X is O.

Materials that are needed to produce the monomers disclosed and/or claimed by Applicants in this specification can be purchased from companies such as: (Oakwood chemicals 730 Columbia Hwy. N, Estill, SC 29918); 4-bromophenol, 1,1,1-tris(4-hydroxylphenyl) ethane (TCI America 9211 North Harborgate Street Portland, OR 97203); polyethylene glycol (PEG-200), bisphenol A, bisphenol AF, eugenol, allyl alcohol, potassium fluoride, calcium carbonate, lithium carbonate, potassium carbonate, and cesium carbonate (Alfa-Aesar, 2 Radcliff Rd, Tewksbury, MA 01876); diethylether; 2H,3H-perfluoropentane (Vertrel™ Chemours, The Chemours Company, 1007 Marchket Street P.O. Box 2047, Wilmington, Delaware 19899); perfluoropyridine (SynQuest, SynQuest Laboratories, Inc., 13201 Rachael Blvd, Rt 2054, Alachua FL 32615); L-9939 perfluoropolyether diol (MACH I Inc., 340) E Church Rd, King of Prussia, PA 19406); Krytox® methylene alcohol (Chemours, The Chemours Company, 1007 Marchket Street P.O. Box 2047, Wilmington, Delaware 19899).

### EXAMPLES

The following examples illustrate particular properties and advantages of some of the embodiments of the present invention. Furthermore, these are examples of reduction to practice of the present invention and confirmation that the principles described in the present invention are therefore valid but should not be construed as in any way limiting the scope of the invention.

Example 1: Synthesis of 2-((perfluoropyridin-4-yl)amino)ethyl methacrylate (1). 2-((perfluoropyridin-4-yl)amino)ethan-1-ol was synthesized using a previously published method.₁ A 500 mL r.b. flask equipped with a 25 mL addition funnel, was charged with of 2-((perfluoropyridin-4-yl)amino)ethan-1-ol (9.32 g, 44.4 mmol), triethylamine (7.40 mL, 53.4 mmol), and anhydrous diethyl ether (300 mL). The solution was cooled in an ice bath under nitrogen. To the addition funnel was added anhydrous diethyl ether (10 mL) and methacrylol chloride (5.20 mL, 53.2 mmol). While stirring under nitrogen in the ice bath, the methacrylol chloride solution was added dropwise to the r.b. flask. The suspension was allowed to gradually warm to room temperature and it was stirred under nitrogen for 96 hours. The suspension was vacuum filtered, and the supernatant was concentrated under reduced pressure. The residue was re-crystallized from warm hexanes and 2-((perfluoropyridin-4-yl)amino)ethan-1-ol was obtained as a white crystalline solid (7.49 g, 60.7%): m.p. 62-63 C; ¹H NMR (500 MHz, CDCl₃): δ 6.01 (s, vinyl, 1H), 5.61 (s, vinyl, 1H), 4.90 (bs, —NHCH₂CH₂O—, 1H), 4.38 (t, —NHCH₂CH₂O—, 2H, ³J=5.0 Hz), 3.86 (q, —NHCH₂CH₂O—, 2H, ³J=5.3 Hz), 1.93 (s, CH_{2═}C(CH₃)—, 3H); ¹⁹F NMR (471 MHz, CDCl₃): δ −93.6 (bs, 2F),−163.4 (bs, 2F); ¹³C NMR (125 MHz, CDCl₃): δ 167.8 (C═O), 135.8 (CH₂═C(CH₃)-), 126.4 (CH_{2═}C(CH₃)-), 63.7 (—OCH₂CH₂NH—), 44.1 (—OCH₂CH₂NH—), 18.3 CH₂═C(CH₃)-).

Example 2: FPs made of 1 and methyl methacrylate by radical chain growth techniques. A 4 mL glass sample vial was charged with 1 (100-25 wt %), methyl methacrylate (MMA, 0-75 wt %), 5.0 wt % of azobisisobutyronitrile (AIBN), and THF (1 mL), according to the amounts presented in Table 1. The vial was sealed and gently heated (55° C.) overnight. The resulting polymers were obtained by dissolving the residue in a minimal amount of THF and precipitating into cold MeOH. The isolated polymers were vacuum filtered and dried for 96 hours in a vacuum oven before analyzing by thermogravimetric analysis (TGA), differential scanning calorimetry (DSC), and gel-permeation chromatography (GPC), ¹H NMR, ¹⁹F NMR, and FT-IR. Table 1 summarizes the thermal and molecular weight data.

TABLE 1
TGA, DSC, and GPC data summary for Poly 1-4.

| | 1 | MMA | Recovery | T_{g} | T_{Onset} | Mₙ | M_{w} | |
|---|---|---|---|---|---|---|---|---|
| Polymer | (wt %) | (wt %) | (%) | (° C.)* | (° C.)\hat{ } | (Da)^{#} | (Da)^{#} | Ð^{#} |
| 1 | 100 | 0 | 79 | 81.5 | 301.2 | 33,540 | 160,410 | 4.8 |
| 2 | 75 | 25 | 86 | 93.9 | 287.5 | 21,270 | 65,290 | 3.1 |
| 3 | 50 | 50 | 84 | 104.0 | 279.6 | 20,600 | 50,570 | 2.5 |
| 4 | 25 | 75 | 53 | 109.5 | 271.5 | 17,980 | 37,990 | 2.1 |

*DSC conditions: cycled −80° C. to 180° C., data reported is from the third cycle, analysis performed under N₂.
\hat{ }TGA conditions: ramp 10° C./min, ambient to 800° C., temperature at 5% weight loss under N₂.
^{#}GPC conditions: analysis performed in THF utilizing calibration with polystyrene standards.

Example 3: FPs made of 1 and methyl methacrylate using 2-cyano-2-propyl benzodithioate and reversible addition-fragmentation chain-transfer methods. A 25 mL r.b. flask was charged with 1 (100-25 wt %), MMA (0-75 wt %), AIBN (25 mg, 2.5 wt %), 2-cyano-2-propyl benzodithioate (15 mg, 1.5 wt %), and benzene (4 mL), according to the amounts presented in Table 2. Following, the r.b. flask was equipped with a magnetic stir bar, a fin denser, and a rubber stopper. Nitrogen was passed through the vessel for 10 minutes to allow for positive nitrogen flow; a second needle was added to the rubber stopper for the exchange of air and nitrogen. The secondary needle was removed after 10 minutes, and the vessel was further purged with nitrogen for another 5 minutes. The RBF was heated to 60° C., and the contents were left to react for approximately 48 hours. Volatiles were removed under reduced pressure, and the resulting polymers were obtained by dissolving the residue in a minimal amount of THF and precipitating into cold MeOH. The isolated polymers were vacuum filtered and dried for 48 hours in a vacuum oven before analyzing by thermogravimetric analysis (TGA), differential scanning calorimetry (DSC), and gel-permeation chromatography (GPC), ¹H NMR, ¹⁹F NMR, and FT-IR. Table 2 summarizes the thermal and molecular weight data.

TABLE 2
TGA, DSC, and GPC data summary for Poly 5-8.

| | 1 | MMA | Recovery | T_{g} | T_{Onset} | Mₙ | M_{w} | |
|---|---|---|---|---|---|---|---|---|
| Polymer | (wt %) | (wt %) | (%) | (° C.)* | (° C.)\hat{ } | (Da)^{#} | (Da)^{#} | Ð^{#} |
| 5 | 100 | 0 | 16 | 103 | 256 | 36,970 | 85,050 | 2.3 |
| 6 | 75 | 25 | 79 | 97 | 243 | 13,010 | 26,450 | 2.0 |
| 7 | 50 | 50 | 88 | 86 | 184 | 12,550 | 28,510 | 2.3 |
| 8 | 25 | 75 | 77 | 78 | 175 | 10,530 | 22,500 | 2.1 |

*DSC conditions: cycled −10° C. to 120° C., data reported is from the third cycle, analysis performed under N₂.
\hat{ }TGA conditions: ramp 10° C./min, ambient to 600° C., temperature at 5% weight loss under N₂.
^{#}GPC conditions: analysis performed in THF utilizing calibration with polystyrene standards.

Example 4: FPs made of 1 and methyl methacrylate using 2-(dodecylthiocarbonothioylthio)-2-methylpropionic acid and reversible addition-fragmentation chain-transfer methods. The procedure developed for Poly 5-8 was followed for Poly 9-12 except 2-(dodecylthiocarbonothioylthio)-2-methylpropionic acid was used in place of 2-cyano-2-propyl benzodithioate. Table 3 summarizes the thermal and molecular weight data obtained.

TABLE 3
TGA, DSC, and GPC data summary for Poly 9-12.

| | 1 | MMA | Recovery | T_{g} | T_{Onset} | Mₙ | M_{w} | |
|---|---|---|---|---|---|---|---|---|
| Polymer | (wt %) | (wt %) | (%) | (° C.)* | (° C.)\hat{ } | (Da)^{#} | (Da)^{#} | Ð^{#} |
| 9 | 100 | 0 | 44 | 57.3 | 272.0 | 31,300 | 104,780 | 3.3 |
| 10 | 75 | 25 | 66 | 74.0 | 263.2 | 24,570 | 61,530 | 2.5 |
| 11 | 50 | 50 | 81 | 88.9 | 331.8 | 18,030 | 41,240 | 2.3 |
| 12 | 25 | 75 | 83 | 88.7 | 294.7 | 20,160 | 47,600 | 2.4 |

*DSC conditions: cycled −10° C. to 120° C., data reported is from the third cycle, analysis performed under N₂.
\hat{ }TGA conditions: ramp 10° C./min, ambient to 600° C., temperature at 5% weight loss under N₂.
^{#}GPC conditions: analysis performed in THF utilizing calibration with polystyrene standards.

Example 5: FPs made of 1 and methacrylic acid using 2-(dodecylthiocarbonothioylthio)-2-methylpropionic acid and reversible addition-fragmentation chain-transfer methods. The procedure developed for Poly 9-12 was followed for Poly 13-16 except methacrylic acid (MAA) was used in place of MMA. Table 4 summarizes the thermal and molecular weight data obtained.

TABLE 4
TGA, DSC, and GPC data summary for Poly 13-16.

| | 1 | MMA | Recovery | T_{g} | T_{Onset} | Mₙ | M_{w} | |
|---|---|---|---|---|---|---|---|---|
| Polymer | (wt %) | (wt %) | (%) | (° C.)* | (° C.)\hat{ } | (Da)^{#} | (Da)^{#} | Ð^{#} |
| 13 | 100 | 0 | 44 | 57.34 | 272.0 | 31,300 | 104,780 | 3.3 |
| 14 | 75 | 25 | 84 | 131.13 | 365.39 | 25,470 | 109,630 | 4.3 |
| 15 | 50 | 50 | 92 | 121.41 | 372.81 | 22,780 | 76,640 | 3.4 |
| 16 | 25 | 75 | 94 | 124.25 | 385.03 | 8,880 | 14,920 | 1.7 |

*DSC conditions: cycled −10° C. to 200° C., data reported is from the third cycle, analysis performed under N₂.
\hat{ }TGA conditions: ramp 10° C./min, ambient to 600° C., temperature at 5% weight loss under N₂.
^{#}GPC conditions: analysis performed in THF utilizing calibration with polystyrene standards.

Example 6: FPs made of 1 and methacrylic acid by radical chain growth techniques. The procedure developed for Poly 1-4 was followed for Poly 17-19 except methacrylic acid (MAA) was used in place of MMA. Table 5 summarizes the thermal and molecular weight data obtained.

TABLE 5
TGA, DSC, and GPC data summary for Poly 17-19.

| | 1 | MMA | Recovery | T_{g} | T_{Onset} | Mₙ | M_{w} | |
|---|---|---|---|---|---|---|---|---|
| Polymer | (wt %) | (wt %) | (%) | (° C.)* | (° C.)\hat{ } | (Da)^{#} | (Da)^{#} | Ð^{#} |
| 17 | 75 | 25 | 87 | 112.9 | 399.0 | 52,540 | 208,270 | 4.0 |
| 18 | 50 | 50 | 76 | 114.5 | 383.6 | 70,110 | 225,020 | 3.1 |
| 19 | 25 | 75 | 58 | 115.0 | 370.1 | 43,830 | 104,312 | 2.4 |

*DSC conditions: cycled −80° C. to 180° C., data reported is from the third cycle, analysis performed under N₂.
\hat{ }TGA conditions: ramp 10° C./min, ambient to 800° C., temperature at 5% weight loss under N₂.
^{#}GPC conditions: analysis performed in THF utilizing calibration with PMMA.

Example 7: FPs made of 1 and methacrylic acid using 2-cyano-2-propyl benzodithioate and reversible addition-fragmentation chain-transfer methods. The procedure developed for Poly 5-8 was followed for Poly 20-22 except methacrylic acid (MAA) was used in place of MMA. Table 6 summarizes the thermal and molecular weight data obtained.

TABLE 6
TGA, DSC, and GPC data summary for Poly 20-22.

| | 1 | MMA | Recovery | T_{g} | T_{Onset} | Mₙ | M_{w} | |
|---|---|---|---|---|---|---|---|---|
| Polymer | (wt %) | (wt %) | (%) | (° C.)* | (° C.)\hat{ } | (Da)^{#} | (Da)^{#} | Ð^{#} |
| 20 | 75 | 25 | 78 | 122 | 232 | 28,840 | 131,040 | 4.5 |
| 21 | 50 | 50 | 94 | 121 | 215 | 14,490 | 70,090 | 4.8 |
| 22 | 25 | 75 | 96 | 120 | 169 | 16,190 | 31,413 | 1.9 |

*DSC conditions: cycled −10° C. to 200° C., data reported is from the third cycle, analysis performed under N₂.
\hat{ }TGA conditions: ramp 10° C./min, ambient to 600° C., temperature at 5% weight loss under N₂.
^{#}GPC conditions: analysis performed in THF utilizing calibration with PMMA.

Example 8: FPs made of 1 and methyl methacrylate using 2-cyano-2-propyl benzodithioate and reversible addition-fragmentation chain-transfer methods. The procedure developed for Poly 5-8 was followed for Poly 23-26 except 0.35 wt % AIBN and 1.5 wt % of 2-cyano-2-propyl benzodithioate was used. Table 7 summarizes the thermal and molecular weight data obtained.

TABLE 7
TGA, DSC, and GPC data summary for Poly 23-26.

| | 1 | MMA | Recovery | T_{g} | T_{Onset} | Mₙ | M_{w} | |
|---|---|---|---|---|---|---|---|---|
| Polymer | (wt %) | (wt %) | (%) | (° C.)* | (° C.)\hat{ } | (Da)^{#} | (Da)^{#} | Ð^{#} |
| 23 | 100 | 0 | 73 | 73 | 195 | 8,330 | 10,400 | 1.2 |
| 24 | 75 | 25 | 40 | 91 | 205 | 15,000 | 20,220 | 1.3 |
| 25 | 50 | 50 | 37 | 97 | 232 | 10,760 | 14,510 | 1.3 |
| 26 | 25 | 75 | 67 | 112 | 258 | 11,460 | 13,800 | 1.2 |

*DSC conditions: cycled −10° C. to 150° C., data reported is from the third cycle, analysis performed under N₂.
\hat{ }TGA conditions: ramp 10° C./min, ambient to 600° C., temperature at 5% weight loss under N₂.
^{#}GPC conditions: analysis performed in THF utilizing calibration with PMMA.

Methods: Differential Scanning calorimetry (DSC) data was collected on a TA instrument DSC2500. Samples (5-15 mg) were sealed in aluminum hermetic pans with an empty sealed aluminum hermetic pan serving as the reference. Data was cycled at a rate of 10° C./min under N₂. Data reported is from the third cycle. Thermogravimetric Analysis (TGA) data was collected on a TA instrument SDT650 or TGA5500. Samples for the SDT650 (5-15 mg) were analyzed in a 90 μL alumina crucible at a rate of 10° C./min under N₂. Samples for the TGA5500 (5-15 mg) were measured with a 100 μL platinum pan at a rate of 10° C./min under N₂. All thermal analysis was performed using TRIOS software. Gel Permeation Chromatography (GPC) analysis was performed on a Polymer Laboratories GPC 220 with a RI detector. Samples (1-2 mg/mL) were eluted in series through a Polymer Labs PLgel Mixed-B LS column at 40° C. using a flowrate of 1mL/min. Samples were eluted in THF (HPLC grade) using polystyrene or polymethyl methacrylate standards (Infinity Lab EasiVial PS-H or EasiVial PM). Data was analyzed using Agilent GPC/SEC software. FT-IR spectra were collected on a ThermoFisher Scientific Nicolet iS10 or iS20 FT-IR spectrometer. Data was collected in % transmittance, after 16 scans, and analyzed using OMNIC software. ¹H (500 MHz) and ¹⁹F NMR (470 MHz) spectra were collected on a JEOL-ECX-500R (500 MHz) spectrometer. ¹H spectra were referenced to TMS or appropriate NMR solvent (THF-d₈, acetone-d₆, acetonitrile-d₃, or dimethyl sulfoxide-d₆) and recorded after 8 or 16 scans. An x-sweep of 0 or 5ppm was used. ¹⁹F NMR were referenced to CFCl₃ and recorded after 8 or 16 scans. Chemical shifts were reported on the ppm scale.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the scope of the general inventive concept.

What is claimed is:

1. A monomer having the following formula: 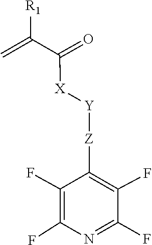
wherein
R₁ is H or a hydrocarbon;
X is O, NH, or S;
Y is a hydrocarbon; and
Z is O, NH, or S.

2. The monomer of claim 1 wherein:
R₁ is H, methyl, ethyl, propyl, iso-propyl, n-butyl, tert-butyl, sec-butyl,
n-pentyl, n-hexyl, n-hepyl, n-octyl, or phenyl; and
Y is ethyl, propyl, iso-propyl, n-butyl, tert-butyl, sec-butyl,
n-pentyl, n-hexyl, n-hepyl, n-octyl, or phenyl.

3. The monomer of claim 2 wherein: R₁ is H or methyl, Y is ethyl or phenyl, X is O.

4. A polymer comprising the monomer of claim 1.

5. The polymer of claim 4, said polymer having Structure 1 or Structure 2 below: 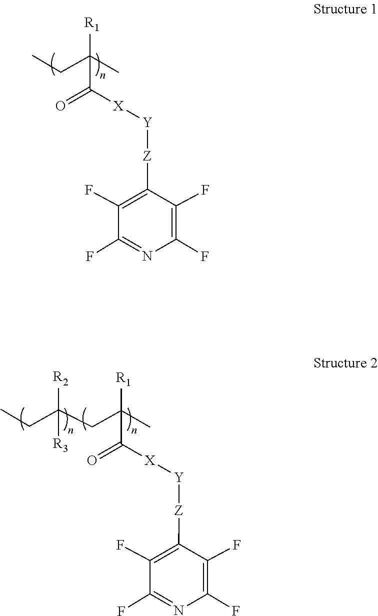
a) wherein for Structure 1
R₁ is H or a hydrocarbon;
X=O, NH, or S;
Y=a hydrocarbon;
Z=O, NH, or S; and
the indice n is an integer from 20 to 150; and
b) wherein for Structure 2
R₁, R₂, or R₃ are each independently H or a hydrocarbon;
X=O, NH, or S;
Y=a hydrocarbon;
Z=O, NH, or S; and
the indice n is an integer from 20 to 150.

6. The polymer of claim 5 wherein
a) for Structure 1 R₁ is H, methyl, ethyl, propyl, iso-propyl, n-butyl, tert-butyl, sec-butyl, n-pentyl, n-hexyl, n-hepyl, n-octyl, or phenyl; and
Y is ethyl, propyl, iso-propyl, n-butyl, tert-butyl, sec-butyl, n-pentyl, n-hexyl, n-hepyl, n-octyl, or phenyl; and
b) for Structure 2:
R₁ is H, methyl, ethyl, propyl, iso-propyl, n-butyl, tert-butyl, sec-butyl, n-pentyl, n-hexyl, n-hepyl, n-octyl, or phenyl;
R₂ and are R₃ are each independently H, methyl, ethyl, propyl, iso-propyl, n-butyl, tertbutyl, sec-butyl, n-pentyl, n-hexyl, n-hepyl, n-octyl, phenyl, O(C═O)CH₃, O(C═O)OH, Cl, F, CN; and
Y is ethyl, propyl, iso-propyl, n-butyl, tert-butyl, sec-butyl, n-pentyl, n-hexyl, n-hepyl, n-octyl, or phenyl.

7. The polymer of claim 6 wherein
a) for Structure 1 R₁ is H or methyl, Y is ethyl or phenyl, and X is O; and
b) for Structure 2 R₁ is H or methyl, R₂ is H or methyl, R₃ is O(C═O)CH₃, Y is ethyl or phenyl, and X is O.

8. The polymer of claim 4, said polymer having a number average molecular weight of from about 5,000 Da to about 50,000 Da.

9. The polymer of claim 4, said polymer having a number average molecular weight of from about 8,000 Da to about 75,000 Da.

10. The polymer of claim 4, said polymer having a polydispersity of from about 1.2 to about 4.8.

11. An article comprising the polymer of claim 4.

12. The article of claim 11, said article being a textile, or a membrane.

13. A process of making a monomer comprising reacting a monomeric precursor having Structure 1 and an acrylol chloride having Structure 2 in the presence of an amine base, preferably said amine base is triethylamine 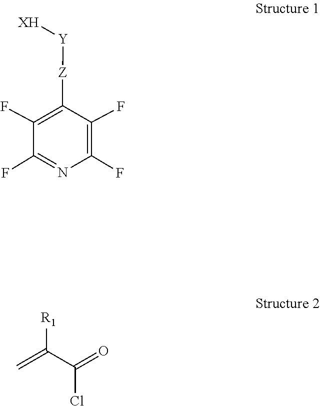
a) wherein for Structure 1
X=O, NH, or S;
Y=a hydrocarbon; and
Z=O, NH, or S; and
b) for Structure 2 R₁ is H or a hydrocarbon.

14. The process of claim 13 wherein:
a) for Structure 1 Y is ethyl, propyl, iso-propyl, n-butyl, tert-butyl, sec-butyl, n-pentyl, n-hexyl, n-hepyl, n-octyl, or phenyl; and
b) for Structure 2 R₁ is H, methyl, ethyl, propyl, iso-propyl, n-butyl, tert-butyl, sec-butyl, n-pentyl, n-hexyl, n-hepyl, n-octyl, or phenyl.

15. The process of claim 13 wherein for Structure 1 Y is ethyl or phenyl, X is O; and for Structure 2 R₁ is H or methyl.

16. A process of making a polymer comprising polymerizing a monomer having Structure 3 below: 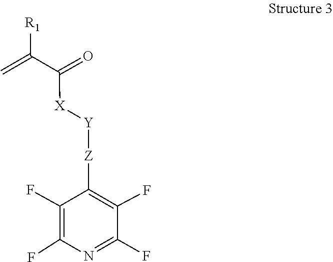
wherein
R₁ is H or a hydrocarbon;
X=O, NH, or S;
Y=a hydrocarbon; and
Z=O, NH, or S.

17. The process of claim 16 wherein for said monomer having Structure 3:
R₁ is H, methyl, ethyl, propyl, iso-propyl, n-butyl, tert-butyl, sec-butyl, n-pentyl, n-hexyl, n-hepyl, n-octyl, or phenyl; and
Y=ethyl, propyl, iso-propyl, n-butyl, tert-butyl, sec-butyl, n-pentyl, n-hexyl, n-hepyl, n-octyl, or phenyl.

18. The process of claim 16 wherein for said monomer having Structure 3 R₁ is H or methyl, Y is ethyl or phenyl, X is O.
